SEQUENCE LISTING (11 sequences)

SEQ ID NO: 1 - protein, 15 aa, Gadus
  trypsin
IVGGYQRSQQVSLNS

SEQ ID NO: 2 - protein, 19 aa, bovine
VGGYTCGANTVPYQVSLNS

SEQ ID NO: 3 - protein, 14 aa, Unknown
  hydrobionts
IVGGVGIYQLSLQD

SEQ ID NO: 4 - protein, 14 aa, Unknown
  hydrobionts
IVGGVGIYQLSFQD

SEQ ID NO: 5 - protein, 15 aa, Unknown
  hydrobionts
IVGGQSGSWQVGLFF

SEQ ID NO: 6 - protein, 15 aa, Unknown
  hydrobionts
IVGGSSGQFYQSFQD

SEQ ID NO: 7 - protein, 12 aa, Unknown
  hydrobionts
IVGGQWVQVLFI

SEQ ID NO: 8 - protein, 11 aa, Unknown
  hydrobionts
IVGGQVQVLFI

SEQ ID NO: 9 - protein, 11 aa, Unknown
  hydrobionts
DYDYDIQLKGL

SEQ ID NO: 10 - protein, 14 aa, Unknown
  hydrobionts
FDNYNFINSILDGV

SEQ ID NO: 11 - protein, 15 aa, Unknown
  hydrobionts
ILGDYLSGGVVYVFG

(19) United States
(12) Patent Application Publication
(10) Pub. No.: US 2012/0058103 A1
(43) Pub. Date: Mar. 8, 2012

(54) COMPOSITION OF INDIVIDUAL PROTEOLYTIC ENZYMES

(75) Inventor: Olga A. KLIMOVA, Moscow (RU)

(73) Assignee: JOINT-STOCK COMPANY 'HIGH TECH', St. Petersburg (RU)

(21) Appl. No.: 12/487,463

(22) Filed: Jun. 18, 2009

Related U.S. Application Data
(63) Continuation of application No. PCT/RU2007/000677, filed on Dec. 3, 2007.

(30) Foreign Application Priority Data
Dec. 20, 2006 (RU) 2006147331

(51) Int. Cl. A61K 8/66; A61P 17/02; A61Q 19/00; C12N 9/50; A61K 38/48

(52) U.S. Cl. 424/94.63; 435/219

(57) ABSTRACT

The invention relates to bioengineering, in particular to producing a composition of individual enzymes exhibiting the extended range of proteolytic activity, thereby enabling the composition to hydrolyse protein substrates up to individual amino acids. The composition contains at least two proteases, the molecular mass of which ranges from 23 to 36 kilodaltons and which have the following N-terminal amino acid sequences: I V G G T E V T P G E I P Y Q L S L Q D-I V G E V T P G E I P Y Q L S F Q D-I V G G Q E A S P G S W P X Q V G L F F-E A T S G Q F P Y Q X S F Q D-I V G G Q E A T P H T W V H Q V A L F E A T P H T X V H Q A L F I-A M D X T A Y X D Y D E I Q A X L K N T F E E I N S I L D G V-A A I L G D E Y L X S G G V V P Y V F G-Medicinal and cosmetic means containing the inventive composition of individual highly purified proteolytic enzymes in the form of an active agent make it possible to reduce the concentration of the composition during the use thereof due to the higher enzymatic activity and do not generate allergic reactions during a long term use. In addition, the composition formulation can be optimised (also standardised) with respect to a patient, thereby increasing the efficiency of the composition and reducing side effects of the use thereof.

### RELATED APPLICATIONS

This application is a Continuation of International Application No. PCT/RU2007/000677, filed on Dec. 3, 2007, which claims priority to Russian Patent Application No. 2006147331, filed on Dec. 20, 2006, both of which are incorporated herein by reference in their entirety.

### FIELD OF THE INVENTION

The invention relates to bioengineering, and in particular to the production of a composition of individual enzymes possessing a wide spectrum of proteolytic activity that enables the composition to hydrolyze protein substrates all the way to individual amino acids.

The composition includes proteolytic enzymes (endo- and exopeptidases) that are distinctly individualized, including the N-terminal sequence and molecular weight, and are characterized by high activity in relation to many peptides and protein substrates, including collagens of many types.

### BACKGROUND OF THE INVENTION

The proteolytic enzymes perform many physiological functions, from the digestion of protein to more specific functions, such as the activation of zymogens and the complement system, and participate in the process of protein coagulation and the lysis of fibrin clots, the maturation of hormones and biologically active peptides from precursor proteins, and the transport of proteins through cell membranes. Comparison of the amino acid sequences, three-dimensional structures, and mechanisms of enzymatic reactions makes it possible to divide proteolytic enzymes into several groups.

The classification of the proteolytic enzymes based on their catalysis mechanism makes it possible to distinguish 4 classes: serine proteases; metalloproteases; thiol (cysteine) and acid (asparagine) proteases. These proteases differ in sensitivity to various inhibitors; for example, serine proteases are inhibited by phenylmethylsulfonyl fluoride (PMSF) and diisopropyl fluorophosphate (DFP); metalloproteases by chelating agents such as EDTA, EGTA, and o-phenantroline; cysteine proteases by iodoacetamide and heavy metals; and asparagine proteases by pepstatin. Serine proteases usually have a weakly-alkaline optimum pH; metalloproteases—neutral; and cysteine proteases and asparagine proteases—acidic.

The rate of hydrolysis of a particular peptide bond by the majority of proteases depends, as a rule, on the substrate specificity of the enzyme, the spatial accessibility of the given peptide bond (especially if a native, and not a denatured, protein is the substrate), and does not depend on the dimensions of the substrate molecule (on the length of the polypeptide chain). Substrate specificity consists in the capacity of a given enzyme to hydrolyze peptide bonds between particular amino acid residues at the highest rate.

The digestive proteases of mammals that have amino acid sequences that are similar to one another, and spatial structures capable of hydrolyzing substrates to small fragments and differing according to the type of cleavable peptide bond, are characteristic representatives of the most numerous class of serine proteases. Thus, chymotrypsin hydrolyzes peptide bonds involving amino acids with aromatic side chains; trypsin, with positively-charged side chains; elastase, with amino acid residues of glycine, and to a lesser extent, of leucine. The mechanism of the observed specificity is determined by differences in the active sites of the proteolytic enzymes.

Proteolytic enzymes possessing narrow substrate specificity are used in molecular biology as tools for the study of the primary structure of proteins and peptides, and the proteases themselves are convenient objects for the study of the structure of proteins and the mechanisms of their functioning as well. Many proteases, such as trypsin, chymotrypsin, papain, and others serve as the active principle of many medications, and are used in cosmetology and veterinary medicine.

Serine proteases isolated from the digestive organs of invertebrates and fishes are known (Zwillirig R., et al., 1975, FEBS Lett. 60, 247-9; Gran G. A., et al., 1981, Methods Erizymol. 80, 722-734; Reeck G. R. and H. Neurath, 1972, Biochem. 11: 503-510; O. A. Klimova, et al., 1990, BBRC, v. 166, No. 3, 1411-1420).

These enzymes' N-terminal amino acid sequences, which are similar to one another, make it possible to classify them as serine proteases. Study of substrate specificity shows that they, in contrast to the digestive proteases of mammals, are capable of hydrolyzing a wider spectrum of substrates, for example, collagens of various types which, due to particular features of amino acid sequence and spatial structure, are resistant to the action of the majority of proteases and are accessible for hydrolysis only by specific enzymes—collagenases.

True collagenases (of tissue and microbial origin) that belong to the class of metalloproteases cleave native collagen at one point of one of the three polypeptide chains of the basic structural element of native collagen—tropocollagen.

By contrast with the true collagenases, serine proteases of invertebrates cleave all three polypeptide chains of tropocollagen, and in addition, the peptides formed undergo further hydrolysis all the way to individual amino acids, which are either included in the process of anabolism or are rapidly excreted from the body without causing intoxication. It is evident that the ability of the serine proteases of invertebrates and fishes to hydrolyze a wide spectrum of substrates is associated with the particular features of the structure not only of their active sites, but with the structure of the substrate-binding sites—the secondary interactions of the substrate with the substrate-binding site make an essential contribution to the increase in catalytic activity. Thus, the activity of these proteases with respect to the hydrolysis of long polypeptides is greater, and of the short synthetic substrates, BAEE, BTEE, and Ac(Ala)3PNA, much less, than for trypsin, chymotrypsin, and elastase respectively.

In a number of cases, however, there is the objective of total proteolysis of various protein substrates all the way to individual amino acids, so that the amino acids obtained in the process could be used as building blocks in the processes of anabolism.

An enzyme preparation, kollaza (Collasum), is known, consisting of a mixture of two isozymes A and C of serine collagenolytic protease (RF Patent No. 2121503 of 1996 Sep. 5, IPC: C12N9/48; 9/64), that possesses necrolytic activity as well as fibrinolytic and thrombolytic properties. However, the mixture of these two proteases cannot completely hydrolyze many polypeptide substrates; for example, collagen fibers are only partially hydrolyzed, and cleansing wounds containing partially damaged collagen fibers by means of kollaza appears problematic.

A mixture of exo- and endopeptidases isolated from Antarctic krill and the use of this mixture in the form of a purified solution was applied for in U.S. Pat. Nos. 4,801,451 and 4,963,491, and the use of these enzymes for the cleansing of purulent necrotic tissue from wounds is applied for in U.S. Pat. No. 4,801,451.

However, unpurified and poorly characterized substances were applied for in these patents. The authors themselves term these “multifunctional proteolytic enzymes,” which by definition signifies their non-specificity and the unknown nature of the mechanism of action.

A mixture of serine proteases obtained from fishes (Atlantic cod), RF patent for invention No. 2264824, is closest to the composition we are applying for. Pharmaceutically and/or cosmetically active compositions containing trypsins and chymotrypsins obtained from cod, and in particular the Atlantic cod, as the active component are applied for in that patent. There are three basic isoforms of trypsin in the Atlantic cod that have already been purified and characterized. They have been named trypsin I, II, and III (Asgeirs son et al., Eur. J. Biochem. 180:85-94, 1989). The cod trypsins have the N-terminal sequence I-V-G-G-Y-Q/E-C-E/T-K/R-H-S-Q-A-H-Q-V-S-L-N-S, whereas the trypsins of mammals, for example, bovine trypsin, have the N-terminal sequence I-V-G-G-Y-T-C-G-A-N-T-V-P-Y-Q-V-S-L-N-S. All three isoforms of cod trypsin have a similar molecular weight of about 24 kD.

However, it is known that serine proteases are endopeptidases, and hydrolyze polypeptide substrates to the state of small peptides, but not to individual amino acids; in addition, they are unable to hydrolyze a number of substrates, for example, native collagen; this narrows the possibilities for future use of such an enzyme composition.

### SUMMARY OF THE INVENTION

The objective of this invention consists in the creation of a composition consisting of proteolytic enzymes that possesses activity, stability in wide temperature and pH ranges, and resistance to autolysis, and intended for extensive use for diagnostic and therapeutic purposes, in cosmetology and pharmacology, as well as in bioengineering.

As a result of the authors' studies, natural complexes of proteolytic enzymes (proteases) with molecular weights of 100-11 kilodaltons were obtained from the digestive organs of hydrobionts. By means of a combination of chromatic methods, individual components with the following N-terminal amino acid sequences, with molecular weights of 23-26 kilodaltons, were isolated and purified to a homogeneous state from the proteolytic complexes:

I V G G T E V T P G E I P Y Q L S L Q D -
I V G G T E V T P G E I P Y Q L S F Q D -
I V G G Q E A S P G S W P X Q V G L F F -
I V G G S E A T S G Q F P Y Q X S F Q D -
I V G G Q E A T P H T W V H Q V A L F I -
I V G G Q E A T P H T X V H Q V A L F I -
A M D X T A Y X D Y D E I Q A X L K G L -
A F D X T N Y N T F E E I N S I L D G V -
A A I L G D E Y L X S G G V V P Y V F G -

The new proteolytic compositions created on the basis of complexes of proteases and/or individual components with molecular weights of 23-26 kilodaltons possess greater enzymatic activity and depth of hydrolysis in relation to many peptide and protein substrates (including collagens of various types) as compared with those previously known.

The above and other features of the invention including various novel details of construction and combinations of parts, and other advantages, will now be more particularly described and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

Examples of the constituents of compositions with the above-enumerated components are presented below.

Components of the composition with molecular weight of 100-37 and 22-11 kDaltons were identified by electrophoresis in polyacrylamide gel (PAAG) under denaturing conditions (in the presence of sodium dodecyl sulfate); the N-terminal amino acid sequences were not determined in connection with the negligible content of the components in the composition. The percentage content of the components in the composition were determined on the basis of staining of the electrophoretogram in PAAG with subsequent scanning of the gel. For this purpose, when the electrophoresis was completed, the gel was fixed in a 5% solution of trichloracetic acid, stained with a solution of Coomassie R-250 using the standard technique (Osterman, L. A. Methods of Investigation of Proteins and Nucleic Acids Electrophoresis and Ultracentrifugation [Practical Handbook], Moscow: Nauka, 1981, 288 pp.), and densitometric analysis was carried out with the aid of the Biometra, Germany (cat. No. 035-300) gel documentation system, which provides computer-aided reading of information on the optical density of the preliminarily stained gel, analysis, and storage of the results.

TABLE 1
Examples of the Makeup of Compositions Based on
Individual Proteolytic Enzymes

| Molecular | | | | |
|---|---|---|---|---|
| Weight of | | | | |
| Individual | | | | |
| Proteolytic | | | | |
| Enzyme | | | | |
| Component of | | | | |
| the | | Content of the Component | | |
| Composition, | N-Terminal Amino Acid | in the Composition, % | | |
| kDalton | Sequence | 1 | 2 | 3 |
| 100-37 | | 0.6 | 0.3 | — |
| 36 | I V G G T E V T P G E I P Y Q L S L Q D - | 6.0 | 11.0 | 13.0 |
| 35-II | I V G G T E V T P G E I P Y Q L S F Q D - | 22.0 | 20.0 | 34.0 |
| 28 | I V G G Q E A S P G S W P X Q V G L F F - | 13.0 | 11.0 | 8.0 |
| 25-I | I V G G S E A T S G Q F P Y Q X S F Q D - | 12.0 | 9.0 | — |
| 25-II | I V G G Q E A T P H T W V H Q V A L F I - | 6.0 | 6.0 | — |
| 25-III | I V G G Q E A T P H T X V H Q V A L F I - | 14.0 | 14.0 | 5.0 |
| 32 | A M D X T A Y X D Y D E I Q A X L K G L - | 6.0 | 4.0 | 40.0 |
| 35-I | A F D X T N Y N T F E E I N S I L D G V - | 4.0 | 4.0 | |
| 23 | A A I L G D E Y L X S G G V V P Y V F G - | 15.0 | 20.0 | |
| 22-11 | | 1.4 | 0.7 | |

In the course of the studies the proposed alternative embodiments of the compositions of proteolytic enzymes demonstrated greater enzymatic activity and the absence of allergic reactions during prolonged use. The advantages of the composition being applied for (in particular, the depth of proteolysis) are confirmed by the results of comparative tests of compositions with different content of the components (as per examples 1 and 3 of Table 1) acting on type I collagen (Table 2). Tests of the composition as per example 2 of Table 1 yielded results that are similar to the results of the composition as per example 1; therefore they are not included in Table 2.

Soluble collagen, 200 μL, from the skin of bovine cattle (Sigma No. C8919) was placed into a plastic test tube of the Eppendorf type with a notched lid, was dialyzed against distilled water for 12 hours at +40° C.; 300 μL of a 0.05 M solution of TES with 0.36 mM CaCl₂, pH 7.5, were added, mixed, and incubated at 37° C. for 15 minutes, following which 10 μL of a solution of the proteolytic enzymes were added. Aliquots of the reaction mixture, 50 μL each, were drawn off by automatic pipette at the time intervals indicated in the table and transferred to plastic test tubes of the Eppendorf type, each containing 10 μL of a 50% (W/V) solution of trichloracetic acid, frozen at −70° C. for 1 hour, and thawed and centrifuged at 5000 g for 5 minutes. The supernatants were carefully and meticulously removed, the pellets were dissolved in 10 μL of a 2% solution of sodium dodecyl sulfate; 20 μL of a buffer solution were added to each sample for preparation of specimens; and the specimens were then analyzed by means of electrophoresis in polyacrylamide gel following the Laemmli method (Nature [1970] 277, 680).

The gel was stained with a 0.2% solution of Coomassie R-250 in a 25% solution of isopropanol and 10% acetic acid, washed with a 7% solution of acetic acid, and dried.

The electrophoretogram was analyzed and the percentage content of the fragments was assessed by the stained gel densitometry method (Procedural Guidelines MUK 4.1/4.2.588-96).

TABLE 2
Effect of the Action of a Composition of Proteolytic Enzymes on Type I Collagen from the
Skin of Bovine Cattle

| | Time of Exposure, min | | | | | | | | | | | | | |
|---|---|---|---|---|---|---|---|---|---|---|---|---|---|---|
| | 5 | | 10 | | 15 | | 30 | | 60 | | 90 | | 120 | |
| Composition No. | Size of Collagen Fragments, kDa | | | | | | | | | | | | | |
| (No. of Example from | 1 | 3 | 1 | 3 | 1 | 3 | 1 | 3 | 1 | 3 | 1 | 3 | 1 | 3 |
| Table 1) | % | | | | | | | | | | | | | |
| 200-350 | 92 | 92 | 88 | 92 | 48 | 88 | 80 | 80 | 0 | 76 | 0 | 70 | 0 | 67 |
| 100-200 | 6 | 8 | 10 | 8 | 22 | 10 | 20 | 12 | 8 | 12 | 0 | 12 | 0 | 10 |
| 70-100 | 2 | 0 | 1 | 0 | 18 | 2 | 34 | 6 | 18 | 6 | 4 | 8 | 0 | 10 |
| 45-70 | 0 | 0 | 1 | 0 | 10 | 0 | 32 | 2 | 31 | 6 | 16 | 6 | 1 | 5 |
| 25-45 | 0 | 0 | 0 | 0 | 1 | 0 | 4 | 0 | 28 | 0 | 24 | 3 | 0 | 6 |
| 10-25 | 0 | 0 | 0 | 0 | 1 | 0 | 1 | 0 | 8 | 0 | 39 | 0 | 12 | 0 |
| 0-10 | 0 | 0 | 0 | 0 | 0 | 0 | 1 | 0 | 7 | 0 | 10 | 1 | 87 | 2 |

The differences in the accumulation over time of the low molecular weight constituents in the hydrolysis products of type I collagen make it possible, by formulating the composition on the basis of individual proteolytic enzymes depending on the specific objective, to regulate the rate and depth of hydrolysis of protein substrates.

Hepatocytes from the human adult and embryo (5-8 weeks) liver were obtained by standard techniques. The pellet obtained was collected and resuspended in a 1% albumin hydrolysate. To count the living cells, 1 mL of the cell suspension was sampled; the content of intact cells was determined by staining with a 0.2% solution of trypan blue and subsequent microscopic examination.

TABLE 3
The Effect of a Composition of Proteolytic Enzymes on Human Hepatocytes.

| | | Proteolytic Enzyme Used for the Disaggregation of | | | | | |
|---|---|---|---|---|---|---|---|
| | | Liver Tissue (Concentration in Solution) | | | | | |
| | | | | | | Composition | |
| | | | | Collagenase | | of Individual | |
| | | Trypsin (Sigma | | (Sigma | | Proteolytic | |
| | | T7409, 0.1% | | C9407), | | Enzymes, | |
| Liver Used to Isolate | | Solution | | 0.05% Solution | | 0.01% Solution | |
| Hepatocytes | | +4° C. | +16° C. | +4° C. | +16° C. | +4° C. | +16° C. |
| Human | Total Yield of | 19.02 | 36.43 | 19.28 | 44.22 | 54.31 | 58.45 |
| Adult Liver | Hepatocytes, % | | | | | | |
| | Content of Living | 54.11 | 41.37 | 70.51 | 47.22 | 88.29 | 70.14 |
| | Cells, % | | | | | | |
| Embryo | Total Yield of | 24.33 | 38.41 | 29.52 | 36.29 | 55.90 | 56.46 |
| (5-8 Weeks) | Hepatocytes, % | | | | | | |
| Liver | Content of Living | 58.62 | 52.49 | 76.11 | 52.12 | 92.22 | 77.72 |
| | Cells, % | | | | | | |

The identified advantages of the new composition of proteolytic enzymes have made it possible to use it in medicine for the correction, treatment, and prevention of the development of pathological (hypertrophic and keloidal) scars of the skin and adhesions, as well as for the treatment of wounds of various origins (wounds, burns, frostbite injuries, ulcers).

The use of an ointment, the composition of the active principle of which includes a preparation of elastase and an acrylic acid-based polymer, to accelerate the healing of wounds by means of the effective removal of eschar is known (U.S. Pat. No. 4,276,281). The elastase was obtained from the pancreas of mammals (specifically, horses). This is a serine protease, with a molecular weight of 25,900 Daltons, consisting of 240 amino acid residues, with a known amino acid sequence (Shotten, D. M., Hartley, B. S.; Biochem J. 131, 643, 1973). The use of elastase is based on its high efficacy for the removal of eschar and other macromolecular debris (residues of damaged cells, partially or completely denatured collagen, etc.) from the surface of burn-injured skin of mammals, thereby accelerating the cleansing of the damaged surface and its preparation for granulation.

The main deficiency of such use of elastase is its low activity in relation to a large number of polypeptide substrates, including collagens of various types, and the shallow hydrolysis of polypeptide substrates (down to large fragments) which must be removed from injured surfaces by other means (mechanical or chemical), thus further traumatizing the patent.

In addition, with frequent application of a component such as elastase to the skin, the latter may cause some illnesses due to its toxicity and allergic action. In this connection, this elastase-based therapeutic agent requires limitation in the duration of use, and for some users who are disposed to allergy, it may be contraindicated entirely.

The medicinal agent closest to the one being applied for is the agent proposed in the invention, “A Means of Treatment of Illnesses Accompanied by the Formation of Pus and/or Necrotic Tissues,” according to RF Patent No. 2149644, IPC: A61K 38/48, priority of 28 Nov. 1997, in which a complex of collagenolytic proteinases with a molecular weight of 10-40 kilodaltons, possessing proteolytic activity and obtained from digestive organs of hydrobionts, is used as the active principle. A deficiency of said agent is the fact that the principal biologically active component is a complex that includes as minor components enzymes that possess nonspecific activity, as a result of which the injurious action of the complex of proteases in relation to living cells is significant. In addition, the makeup of the complex of proteases greatly depends on the physiological cycle of the hydrobionts from which this complex was obtained. Given this, the content of the components of the complex and consequently its properties varies over wide limits; this makes the use of the preparation difficult.

### INDUSTRIAL APPLICABILITY

A medicinal agent containing the proposed composition of proteolytic enzymes, consisting of individual enzymes with a high degree of purity, as the active principle makes it possible as a result of greater enzymatic activity to reduce the concentration of the composition during use and does not lead to allergic reactions during prolonged use. In addition, the makeup of the composition can be optimized (including standardized) with respect to the target of the treatment; this increases its efficacy and reduces side effects due to its use.

### Examples of the Use of the Composition of Individual Proteolytic Enzymes

### Example No. 1

Patient K., 41, came for consultation because of scars on her face which had formed about a year earlier in the course of do-it-yourself treatment of acne with urinotherapy in the form of applications of her own urine. A bullous-necrotic form of erysipelatous inflammation developed as a result.

The use of known preparations for the treatment of cicatricial alterations of the skin of the face had been ineffective. The patient was prescribed out-patient treatment at the department of spa medicine and physiotherapy of the Military Medical Academy, and 3 courses of electrophoresis, 10 sessions each, with the composition of proteolytic enzymes to scars of the skin of the face were carried out. The breaks between courses were 1 month. As a result, the scars became practically imperceptible and their density and intensity of staining decreased as compared with the baseline condition.

### Example No. 2

Patient K., 25, sought help for scars in the region of the forehead and eyelids. It is known from her history that she had been in an automobile accident 3 months earlier, in the course of which she received multiple injuries of the soft tissues of the face by glass shards. The wound of the left upper eyelid was sutured. The patient was troubled by the presence of itching in the area of the scars, the presence of palpable small encapsulated foreign bodies of the skin, and disfigurement.

Fifteen sessions of electrophoresis with a composition of proteolytic enzymes were carried out. Breakdown of the capsules and exit outwards of the glass shards commenced as a result of this treatment. In all more than 20 shards came out. The patient subsequently underwent chemical peeling of moderate intensity with the use of trichloracetic acid. Clearing of the small encapsulated foreign bodies from the face, correction of the scars, as well as restoration of the esthetic appearance of the patient's face, were the final result.

### Example No. 3

Patient P., 21, sought help for cicatricial eversion of the left upper eyelid. She received the injury during an automobile accident 2.5 months earlier. A steadily increasing degree of eversion of the eyelid and conjunctivitis (due to slight drying of the eyeball) were noted from the complaints. Ten sessions of electrophoresis and 10 sessions of phonophoresis with a “composition of proteolytic enzymes” were carried out. In addition, injections of the preparation Collalysine were performed 3 times.

As a result of the treatment, carried out over the course of 2 months, the eyelid began to descend and was virtually closing.

### Example No. 4

Patient Z., 42, sought help for a chronically non-healing wound of the anterior surface of the chest on the left. She had had an operation 2 months earlier—a Halsted mastectomy. Marginal necrosis of the skin flap occurred subsequently. Levosin ointment and the preparation Curiosine were used in topical treatment.

On examination: oblong granulating wound, 3.5×6 cm. The base of wound was filled with pale pink granulations, covered with a fibrin membrane. Marginal epithelialization sluggish.

Dressings with a composition of proteolytic enzymes were applied to the wound surfaces for one week, after which they were changed to dressings with Levosin ointment. Healing of the wound was completed by the end of 10 days.

### SOURCES OF INFORMATION

1. Zwillirig R., et al., 1975, FEBS Lett. 60, 247-9; Gran G. A., et al., 1981, Methods Enzymol. 80, 722-734;
2. Reeck G. R. and H. Neurath. 1972, Biochem. 11: 503-510, O. A. Klimova, et al., 1990, BBRC, v. 166, No. 3, 1411-1420
3. Asgeirsson et al., Eur. J. Biochem. 180:85-94, 1989.
4. Osterman, L. A., Metody issledovaniya belkov i nukleinovykh kislot. Elektroforez i ul'tratsentrifugirovanie (prakticheskoe posobie) [In Russian] [Methods of Investigation of Proteins and Nucleic Acids, Electrophoresis and Ultracentrifugation (Practical Handbook)] [in Russian]. Moscow: Nauka, 1981, 288 pp.
5. Shotten, D. M., Hartley, B. S.; Biochem. J. 131, 643, 1973

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition of individual proteolytic enzymes whose makeup includes at least two proteases with a molecular weight from 23 to 36 kilodaltons and the following N-terminal amino acid sequences:
I V G G T E V T P G E I P Y Q L S L Q D -
I V G G T E V T P G E I P Y Q L S F Q D -
I V G G Q E A S P G S W P X Q V G L F F -
I V G G S E A T S G Q F P Y Q X S F Q D -
I V G G Q E A T P H T W V H Q V A L F I -
I V G G Q E A T P H T X V H Q V A L F I -
A M D X T A Y X D Y D E I Q A X L K G L -
A F D X T N Y N T F E E I N S I L D G V -
A A I L G D E Y L X S G G V V P Y V F G -

2. A composition of individual proteolytic enzymes according to claim 1 in the following makeup:

| | | | |
|---|---|---|---|
| 100-37 | kDa | | 0.6% |
| 36 | kDa | I V G G T E V T P G E I P Y | 6% |
| | | Q L S L Q D - | |
| 35-II | kDa | I V G G T E V T P G E I P Y | 22% |
| | | Q L S F Q D - | |
| 28 | kDa | I V G G Q E A S P G S W P X | 13% |
| | | Q V G L F F - | |
| 25-I | kDa | I V G G S E A T S G Q F P Y | 12% |
| | | Q X S F Q D - | |
| 25-II | kDa | I V G G Q E A T P H T W V H | 6% |
| | | Q V A L F I - | |
| 25-III | kDa | I V G G Q E A T P H T X V H | 14% |
| | | Q V A L F I - | |
| 32 | kDa | A M D X T A Y X D Y D E I Q | 6% |
| | | A X L K G L - | |
| 35-I | kDa | A F D X T N Y N T F E E I N | 4% |
| | | S I L D G V - | |
| 23 | kDa | A A I L G D E Y L X S G G V | 15% |
| | | V P Y V F G - | |
| 22-11 | kDa | | 1.4% |

3. A composition of individual proteolytic enzymes according to claim 1 in the following makeup:

| | | | |
|---|---|---|---|
| 100-37 | kDa | | 0.3% |
| 36 | kDa | I V G G T E V T P G E I P Y | 11% |
| | | Q L S L Q D - | |
| 35-II | kDa | I V G G T E V T P G E I P Y | 20% |
| | | Q L S F Q D - | |
| 28 | kDa | I V G G Q E A S P G S W P X | 11% |
| | | Q V G L F F - | |
| 25-I | kDa | I V G G S E A T S G Q F P Y | 9% |
| | | Q X S F Q D - | |
| 25-II | kDa | I V G G Q E A T P H T W V H | 6% |
| | | Q V A L F I - | |
| 25-III | kDa | I V G G Q E A T P H T X V H | 14% |
| | | Q V A L F I - | |
| 32 | kDa | A M D X T A Y X D Y D E I Q | 4% |
| | | A X L K G L - | |
| 35-I | kDa | A F D X T N Y N T F E E I N | 4% |
| | | S I L D G V - | |
| 23 | kDa | A A I L G D E Y L X S G G V | 20% |
| | | V P Y V F G - | |
| 22-11 | kDa | | 0.7% |

4. A composition of individual proteolytic enzymes according to claim 1 in the following makeup:

| | | | |
|---|---|---|---|
| 36 | kDa | I V G G T E V T P G E I P Y | 13% |
| | | Q L S L Q D - | |
| 35-II | kDa | I V G G T E V T P G E I P Y | 34% |
| | | Q L S F Q D - | |
| 28 | kDa | I V G G Q E A S P G S W P X | 8% |
| | | Q V G L F F - | |
| 25-III | kDa | I V G G Q E A T P H T X V H | 5% |
| | | Q V A L F I - | |
| 32 | kDa | A M D X T A Y X D Y D E I Q | 40% |
| | | A X L K G L - | |

5. A composition of individual proteolytic enzymes according to claim 1, distinguished by the fact that it is capable of hydrolyzing native and completely or partially denatured proteins, including collagens of various types, down to individual amino acids.

6. A medicinal agent, including an active agent in the form of a composition according to claim 1 in an effective quantity, and a pharmaceutically usable vehicle.

7. A cosmetic agent, including a composition according to claim 1 in an effective quantity as the active component.
